(19) United States
(12) Patent Application Publication
(10) Pub. No.: US 2013/0156825 A1
(43) Pub. Date: Jun. 20, 2013

(54) Modafinil Formulations

(71) Applicant: Cephalon, Inc., Frazer, PA (US)

(72) Inventors: Moshe Bentolila, Beer Sheva (IL); Aldo Shusterman, Beer Sheva (IL); Moshe Arkin, Kfar Shmariahu (IL); Joseph Kaspi, Givatayim (IL)

(73) Assignee: CEPHALON, INC., Frazer, PA (US)

(21) Appl. No.: 13/773,016

(22) Filed: Feb. 21, 2013

Related U.S. Application Data
(63) Continuation of application No. 13/097,614, filed on Apr. 29, 2011.
Application No. 13/097,614 is a continuation of application No. 12/552,886, filed on Sep. 2, 2009.
Application No. 12/552,886 is a continuation of application No. 10/720,583, filed on Nov. 24, 2003, now abandoned.

(30) Foreign Application Priority Data
Nov. 26, 2002 (IL) 153098

(51) Int. Cl. A61K 31/165

(52) U.S. Cl. CPC A61K 31/165
USPC 424/400; 514/618; 428/402

(57) ABSTRACT

The invention provides an oral pharmaceutical composition comprising modafinil particles, wherein at least about 5% of said modafinil particles have a diameter greater than 200 microns.

### FIELD OF THE INVENTION

The invention relates to an oral pharmaceutical composition comprising modafinil. The composition comprises modafinil particles, wherein at least 5% of said modafinil particles have a diameter greater than 200 μ. Still, this composition showed dissolution rate and blood levels (after oral administration) comparable with Provigil® tablets of the same strength.

### BACKGROUND OF THE INVENTION

Modafinil, also termed 2-[(diphenylmethyl)sulfinyl]acetamide, is marketed in various countries under brand names such as Provigil®, Modiodal® and Vigil®. It is marketed as tablets containing 100 or 200 milligrams of modafinil. This drug is used for treating conditions of hypersomnia and narcolepsy, namely to improve wakefulness in patients with excessive daytime sleepiness associated with narcolepsy. The drug and its uses were described in the already expired U.S. Pat. No. 4,177,290.

U.S. Pat. No. RE36517 (the “517 U.S. Patent”), is assigned to Cephalon Inc. and discloses the significance of the particle size distribution (PSD) of modafinil. Early safety studies of modafinil tablets, done on healthy human volunteers, did not show any adverse effect on humans in doses up to 4500 milligrams. However, in clinical trials conducted later in the US, serious adverse effects such as elevation of heart rate and increase in the blood pressure were observed in some volunteers, at doses of 800 milligrams. Further investigation showed that tablets made with the “late” material had faster dissolution profile than tablets made from “early” material. Tests done on dogs also showed that tablets prepared from the “late” material had higher blood levels than those made from the “early” material.

The '517 U.S. Patent states that the reason for the differences between the two formulations is related to the PSD of the formulations containing modafinil. Specifically, the “early” batches contained modafinil particles which had a median value of 94-143 μ, and 95% of the particles were smaller than 220-280 μ. The “late” batches (data for 2 batches was reported) had a median value of 31-50 μ and the 95% of the particles were smaller than 110-150 μ. Based on these findings, the '517 U.S. Patent is directed to pharmaceutical composition comprising a substantial homogeneous mixture of modafinil particles wherein at least about 95% of the cumulative total of modafinil particles has a diameter of less than about 200 μ. In addition, the median of these modafinil particles has a median smaller than 60 μ.

The '517 U.S. Patent teaches that the PSD of the modafinil in the tablet plays an important role on its therapeutical effect. The “early” and the “late” products differ in: 1) dissolution rate (in vitro); 2) blood level profiles (in vivo) and 3) occurrence of adverse effects. The differences are attributed to the different PSD of the modafinil used.

Strict adherence to the specification of the PSD in the “late” material was admitted to be very important for the safety (demonstrated by lack of adverse effects) and effectiveness (shown by the blood levels and dissolution rate) of the modafinil tablets. Specifically, Applicants of the '517 U.S. Patent have stated that it is preferable that not more than about 5% of the cumulative total (percent cumulative) of modafinil particles in any one dose provided to a mammal have particle sizes greater than about 200 microns;

### BRIEF DESCRIPTION OF THE DRAWINGS

FIG. 1 shows the dissolution results of tablets containing 200 mg made in accordance with Example 1 in comparison with Provigil® tablets of the same strength.

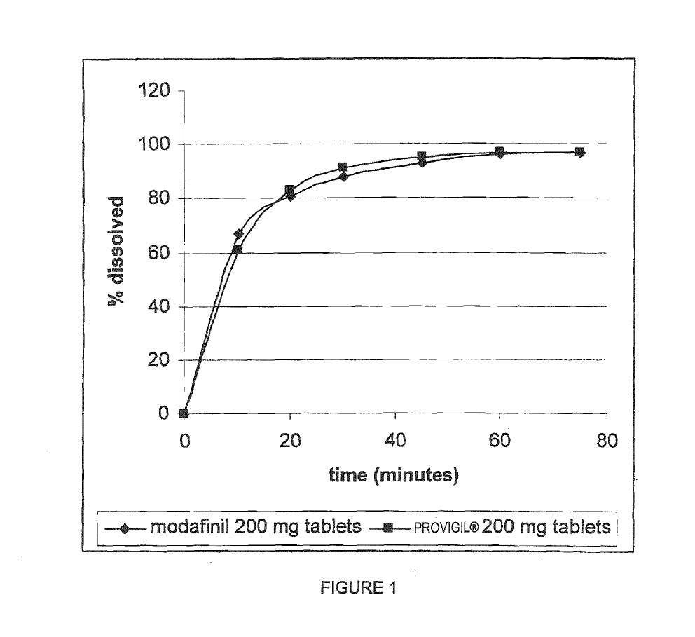

FIG. 2 demonstrates the blood level results of tablets containing 200 mg made in accordance with Example 1 in comparison with Provigil® tablets of the same strength.

### DETAILED EMBODIMENTS OF THE INVENTION

The present invention is directed to modafinil oral and/or pharmaceutical composition, wherein at least 5% of the modafinil particles have a diameter greater than 200 μ.

Surprisingly, we have found there is a way to expand the modafinil specifications of PSD beyond the limits of '517 U.S. patent and still achieve the desirable dissolution rates of Provigil® tablets of the same strength. Moreover, these tablets also showed same blood levels when administered to human volunteers when compared to the blood levels obtained by Provigil® tablets of the same strength. This shows that the modafinil tablets obtained by the present invention, having at least 5% of the modafinil particles greater than 200 μ, are bioequivalent to Provigil® tablets of the same strength, not in agreement with the art taught by the '517 U.S. patent. The '517 U.S. patent predicted significantly lower dissolution rates and significantly lower blood levels for such formulations.

The comparable dissolution rates and blood levels of our composition was achieved using modafinil having large particles (at least 5% larger than 200 μ and median greater than 60 μ) in the presence of dissolution modifiers, usually surfactants.

Although enhancing the dissolution rate (and consequently blood levels) by the addition of dissolution modifiers is a known formulating technique, it is not a trivial issue. There are cases in which this technique helps to improve the solubilization of the drug usually by the addition of a surfactant to the pharmaceutical composition. However, there are instances where adding a dissolution modifier to a formulation do not fulfill the expectation of enhancing solubility of the compound. There are numerous examples of such cases. As examples we can cite B. W. Barry and D. I. El Eini, Journal of Pharmacy and Pharmacology, 28, 210-218 (1976) for dexamethasone; H. Tomida, T. Yotsuyanagi, K. Ikeda, Chemical and Pharmaceutical Bulletin, 26, 2832-2837, (1978) for substituted benzoic acids; L. Bonlokke, I. Hovgaard, H. G. Kristensen, L. Knutson and H. Lennernas, European Journal of Pharmaceutical Sciences, 12, 239-250 (2001) for spironolactone; S. G. Kapsi and J. W. Ayres, International Journal of Pharmacy, 229, 193-203, (2001); and S. R. Levis and P. B. Deasy, International Journal of Pharmacy, 230, 25-33, (2001). These examples show that enhancing the dissolution rate by addition of compounds known to be dissolution modifiers is not obvious.

The ingredients used for the preparation of the modafinil 100 mg and 200 mg tablets according to the present invention are known to be safe and approved for use in oral tablets. They are all described in pharmacopoeial monographs such as USP or NF. The ingredients used for the preparation of the modafinil tablets are colloidal silicon dioxide, crospovidone, lactose, povidone, sodium stearyl fumarate and talc. The modafinil tablets (containing 100 mg and 200 mg modafinil) may be prepared as follows: a mixture of lactose, modafinil and crospovidone may be sprayed by povidone. The granulate may then be dried and milled. Then, the granulate may be mixed with other ingredients. The mixture can be compressed to afford the tablets.

In preferred embodiments of the present invention said composition is further characterized by having a dissolution of more than 50% in 10 minutes.

In especially preferred embodiments of the present invention said composition is further characterized by having a dissolution of more than 80% in 30 minutes.

In one embodiment of the invention at least about 5% of the cumulative total of modafinil particles have a diameter of more than about 250 μ.

In another embodiment of the invention, 10% of the cumulative total of modafinil particles has a diameter of more than about 200 μ.

In another embodiment of the invention, at least about 15% of the cumulative total of modafinil particles has a diameter of more than about 190 μ.

In another embodiment of the invention, the median value of modafinil particles is more than about 60 μ preferably more than 80 μ.

The term “about” in the above PSD characteristics has the meaning of ±20% of the stated value for the percentage of particles above the stated value and ±10% of the stated value of the particle size.

The statistical term “mean” refers hereinafter to the sum of the size measurements of all measurable particles measured divided by the total number of the particles measured. The term “median” indicates that about 50% of all measurable particles measured have a particle size less than the defined median particle size value, and that about 50% of all measurable particles measured have a particle size greater than the defined median particle size value. The term “mode” indicates the most frequent occurring particle size value.

The term “Particle Size Distribution” (PSD) refers to a crystal clear concept when one deals with spherical particles. The results are unique and easy to explain. However, once the particles shape is less similar to spheres, the results are less clear. Different techniques, will give different results. Additionally, in many techniques, the raw data is mathematically manipulated by algorithms to give the PSD results. Different algorithms may also lead to different results.

It has now been found that the modafinil particles used in the present invention are larger than those claimed by the Cephalon patent regardless of the technique used.

Modafinil PSD was measured in '517 U.S. patent by using a Hiac/Royco machine. This machine uses a light obscuration technique for the PSD measurement. We repeated the measurements of the PSD on a Hiac/Royco machine and compared it with a different laser beam obscuration technique offered by the commonly used Malvern machine. Both methods use light obscuration as means to evaluate the PSD, but they work on different principles. The results confirmed the validity of our PSD measurements. Both methods gave similar values. Both methods also gave PSD results that are far away from the scope of the values claimed by the '517 U.S. Patent. The following table summarizes the data. Values are reported in microns. The term D(v,0.5) denotes the diameter of the largest particle found in 50% of the particles sorted in increasing order. The other two terms refer to 85% and 95% of the particles. The terms mean, median and mode are used in their regular statistical meaning. Results are given in table 1.

TABLE 1
PSD of modafinil particles using two measuring techniques

| | | | | | | | D(v, |
|---|---|---|---|---|---|---|---|
| Batch | Method | Mean | Median | mode | D(v, 0.5) | D(v, 0.85) | 0.95) |
| A | Hiac | 51 | 134 | 196 | 133 | 229 | 282 |
| | Malvern | | 107 | | 107 | 235 | 318 |
| B | Hiac | 40 | 104 | 148 | 103 | 194 | 265 |
| | Malvern | | 85 | | 85 | 215 | 293 |
| C | Hiac | 51 | 148 | 193 | 148 | 231 | 277 |
| | Malvern | | 128 | | 128 | 228 | 303 |

Note:
values are in microns.

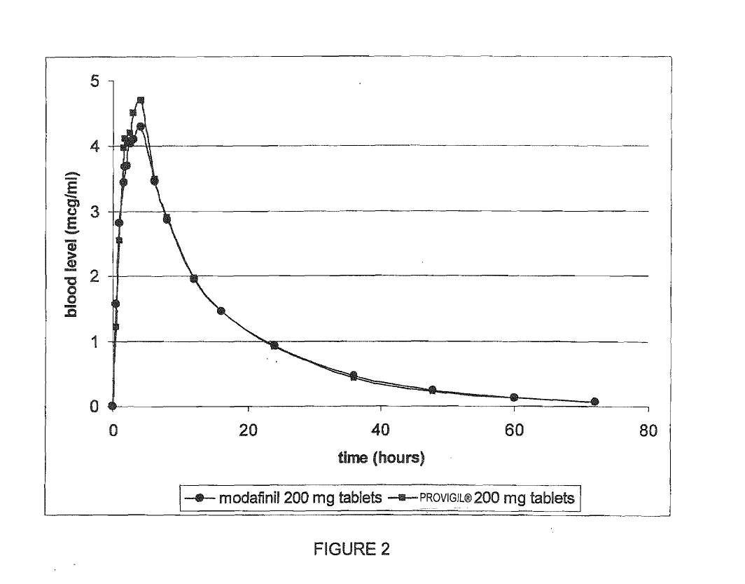

The high similarity between the dissolution rate of Provigil tablets and the tablets made by our new invention is an excellent indication for their bioequivalence. The Physician Desk Reference states that “Absorption of PROVIGIL tablets is rapid, with peak plasma concentrations occurring at 2-4 hours. The bioavailability of PROVIGIL tablets is approximately equal to that of an aqueous suspension.” Since the absorption of modafinil seems not to be a limiting factor, it is safe to assume that similar dissolution rates indicate similar blood levels (hence similar therapeutic effect) for both products. This was verified by the results of the comparative blood levels in human subjects (see FIG. 2).

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLES

### Example 1

Tablets containing 200 mg of modafinil having PSD as provided in table 2 below (values are in microns) were prepared:

TABLE 2
PSD of modafinil used to prepare the modafinil tablets

| Batch | D(v, 0.5) | D(v, 0.85) | D(v, 0.95) | Median |
|---|---|---|---|---|
| A | 107 | 235 | 318 | 107 |
| B | 85 | 215 | 293 | 85 |
| C | 128 | 228 | 303 | 128 |
| D | 95 | 217 | 297 | 95 |

Note:
values are in microns.

Each of the modafinil tablets contained 200 mg modafinil. The following excipients were used: povidone, crospovidone, lactose, colloidal silica, sodium stearyl fumarate and talc.

### Example 2

The tablets described in Example 1 were prepared as follows: a solid mixture of lactose, modafinil and crospovidone was sprayed with an aqueous solution of povidone. The wet granulate was dried and milled. The milled, dry granulate was mixed with the rest of the ingredients and tablets were prepared from the solid mixture in the regular way.

### Example 3

Modafinil 100 mg tablets were prepared according to the formulation and the method of preparing the tablets as described in Examples 1 and 2.

### Example 4

Dissolution rates of the tablets made according to Example 1, were measured in 0.1 N HCl at 37° C. and compared to the dissolution rates of Provigil® tablets 200 mg. The results are summarized in Table 3.

TABLE 3
Comparative dissolution results

% dissolved

| Time (min) | Example 1 tablets | Provigil ® tablets |
|---|---|---|
| 0 | 0 | 0 |
| 10 | 67 | 59 |
| 20 | 82 | 81 |
| 30 | 89 | 90 |
| 45 | 94 | 94 |
| 60 | 97 | 95 |
| 75 | 98 | 96 |

As can be clearly seen, the dissolution rate of the tablets according to Example was similar to the dissolution rate of the Provigil® tablets.

### Example 5

The blood levels of modafinil 200 mg tablet and Provigil® 200 mg tablets were compared in a regular bioequivalence study. The average blood levels (in μg/ml) of 10 human volunteers are given in table 4.

TABLE 4
comparative blood levels results

| | | |
|---|---|---|
| Time after | Modafinil 200 mg | Provigil 200 mg |

| administration | average | Standard | average | standard |
|---|---|---|---|---|
| (hours) | (μg/ml) | deviation | (μg/ml) | deviation |
| 0 | 0 | | 0 | |
| 0.5 | 1.56 | 1.19 | 1.23 | 1.26 |
| 1 | 2.83 | 1.82 | 2.54 | 1.51 |
| 1.5 | 3.44 | 1.85 | 2.54 | 1.51 |
| 1.75 | 3.68 | 1.77 | 4.11 | 2.06 |
| 2 | 3.70 | 1.44 | 4.09 | 1.98 |
| 2.25 | 4.05 | 1.38 | 4.09 | 1.90 |
| 2.5 | 4.04 | 1.29 | 4.19 | 1.67 |
| 3 | 4.10 | 1.30 | 4.51 | 1.50 |
| 4 | 4.29 | 1.35 | 4.69 | 1.16 |
| 6 | 3.46 | 0.79 | 3.49 | 0.69 |
| 8 | 2.87 | 0.59 | 2.90 | 0.61 |
| 12 | 1.96 | 0.43 | 1.98 | 0.35 |
| 16 | 1.46 | 0.31 | 1.46 | 0.26 |
| 24 | 0.94 | 0.26 | 0.91 | 0.17 |
| 36 | 0.47 | 0.16 | 0.43 | 0.13 |
| 48 | 0.24 | 0.11 | 0.23 | 0.10 |

It can be clearly seen that the blood level of modafinil at predetermined time periods after administering the tablets according to Example 1 was similar to the blood level after administering the Provigil® tablets at the same time periods.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. An oral pharmaceutical composition comprising modafinil particles, wherein at least about 15% of the cumulative total of said modafinil particles have a diameter of more than about 200 microns and more than about 5% of the cumulative total of said modafinil particles have a diameter of more than about 250 microns.

2. The pharmaceutical composition of claim 1, wherein the median of the cumulative total of modafinil particles is greater than 60 microns.

3. The pharmaceutical composition of claim 2, wherein the median of the cumulative total of modafinil particles is greater than about 80 microns.

4. The pharmaceutical composition of claim 1, wherein the composition comprises 100 milligrams of said modafinil.

5. The pharmaceutical composition of claim 1, wherein said composition comprises about 200 milligrams of said modafinil.

6. The pharmaceutical composition of claim 1, wherein said composition has a dissolution rate in 0.1 N HCl at 37° C. of more than 80% in 30 minutes.

7. The pharmaceutical composition of claim 1, wherein said composition additionally comprises colloidal silicon dioxide, crospovidone, lactose, talc, sodium stearyl fumarate and povidone.
